(19) United States
(12) Patent Application Publication
(10) Pub. No.: US 2019/0010558 A1
(43) Pub. Date: Jan. 10, 2019

(54) METHOD FOR DETERMINING THE RISK OF RECURRENCE OF AN ESTROGEN RECEPTOR-POSITIVE AND HER2-NEGATIVE PRIMARY MAMMARY CARCINOMA UNDER AN ENDOCRINE THERAPY

(71) Applicant: Myriad International GmbH, Cologne (DE)

(72) Inventors: Karsten Weber, Cologne (DE); Marsel Scheer, Cologne (DE); Christoph Petry, Cologne (DE)

(21) Appl. No.: 16/124,915

(22) Filed: Sep. 7, 2018

Related U.S. Application Data
(63) Continuation of application No. PCT/EP2017/055601, filed on Mar. 9, 2017.

(30) Foreign Application Priority Data
Mar. 9, 2016 (EP) 16159481.7

(51) Int. Cl. C12Q 1/6886

(52) U.S. Cl. CPC C12Q 1/6886; C12Q 2600/158; C12Q 2600/118

(57) ABSTRACT

Provided herein are methods for predicting a result relating to breast cancer in a patient, the method comprising (a) determining the RNA expression levels of three or more of the following 8 genes in a tumor sample from the patient: UBE2C, BIRC5, DHCR7, STC2, AZGP1, RBBP8, IL6ST and MGP; and (b) mathematically combining the expression level values for the genes of the mentioned set to obtain a combined score, the combined score indicating a prognosis for the patient, wherein the RNA expression level values have at least in part not been normalized before the mathematical combination.

This application claims priority International Application No. PCT/EP2017/055601, filed Mar. 9, 2017, which claims priority benefit to EP 16159481.7, filed Mar. 9, 2016, the entire contents of which are hereby incorporated by reference.

### BACKGROUND

The invention relates to a method for predicting a result relating to breast cancer in an estrogen receptor-positive and HER2-negative tumor in a breast cancer patient.

The EndoPredict® score (EP score) is a multivariate score for determining the risk of remote metastases in patients with an estrogen receptor-positive and HER2-negative primary mammary carcinoma under a sole adjuvant endocrine therapy (Filipits et al. Clin. Cancer Res. 17:6012-20 (2011)): A new molecular predictor of distant recurrence in ER-positive, HER2-negative breast cancer adds independent information to conventional clinical risk factors. Clinical Cancer Research 17: 6012-6020; EP 2 553 118 B1). The EP score is a numerical measure of the relative risk that the tumor of the breast cancer patient examined with this EP score will develop remote metastases within 10 years. The determined risk thus can be used to support the decision whether breast cancer patients should be treated with chemotherapy, or whether a milder hormone therapy is sufficient as a treatment. Patients with a relative risk of metastases under an endocrine therapy of more than 10% usually undergo chemotherapy. If the risk of metastases is lower, most physicians recommend the milder hormone therapy. The present invention fulfills the need for advanced methods for the prognosis of breast cancer.

### SUMMARY

In an embodiment, a method for predicting a result relating to breast cancer in an estrogen receptor-positive and HER2-negative tumor in a breast cancer patient is provided. The method comprises, (a) determining the RNA expression levels of at least 4 of the following 8 genes in a tumor sample from the patient: UBE2C, BIRC5, DHCR7, STC2, AZGP1, RBBP8, IL6ST and MGP; (b) mathematically combining the expression level values for the genes of the mentioned set, the values having been determined in the tumor sample, to obtain a combined score, the combined score indicating a prognosis for the patient, wherein the RNA expression level values have at least in part not been normalized before the mathematical combination. In an embodiment, the at least 4 genes are BIRC5, UBE2C, RBBP8, and IL6ST. In an embodiment, the at least 4 genes are any of the panels described in Table 1. In an embodiment, said mathematically combining the expression levels is effected by using the formula

$$= {\sum }_{i = 1}^{8} - {c}_{i} {x}_{i} - 2 , 432381$$

$$or$$

$$= {\sum }_{i = 1}^{k} - {c}_{i} {x}_{i} + ( 20 + \tilde{r} ) {\sum }_{i = 1}^{k} {c}_{i} + {\sum }_{i = k + 1}^{8} - {c}_{i} {x}_{i} + ( 20 + r ) {\sum }_{i = k + i}^{8} {c}_{i} .$$

In an embodiment, said patient has received endocrine therapy or is contemplated to receive endocrine treatment. In an embodiment, a risk of developing breast cancer recurrence or cancer-related death is predicted. In an embodiment, said expression level is determined as a Messenger-RNA expression level. In an embodiment, said expression level is determined by at least one of a PCR based method, a microarray based method, and a hybridization based method. In an embodiment, said determination of expression levels is in a formalin-fixed paraffin embedded tumor sample or in a fresh-frozen tumor sample. In an embodiment, one, two or more thresholds are determined for said combined score, that discriminate into high and low risk, high, intermediate and low risk, or more risk groups by applying the threshold on the combined score. In an embodiment, a high combined score is indicative of benefit from cytotoxic chemotherapy. In an embodiment, information regarding nodal status of the patient is processed in the step of mathematically combining expression level values for the genes to yield a combined score. In an embodiment, said information regarding nodal status is a numerical value if said nodal status is negative and said information is a different numerical value if said nodal status positive and a different or identical number if said nodal status is unknown.

In another embodiment, a kit is provided for performing a method according the methods described herein. In an embodiment, said kit comprising a set of oligonucleotides capable of specifically binding sequences or to sequences of fragments of the genes in a combination of genes, wherein said combination comprises determining the RNA expression levels of at least 4 of the following 8 genes in a tumor sample from the patient: UBE2C, BIRC5, DHCR7, STC2, AZGP1, RBBP8, IL6ST and MGP. In an embodiment, the at least 4 genes of the kit are BIRC5, UBE2C, RBBP8, and IL6ST. In an embodiment, the at least 4 genes are any of the panels described in Table 1.

In another embodiment, a computer program product is provided. In an embodiment, the computer program product is capable of processing values representative of expression levels of a set of genes, mathematically combining said values to yield a combined score, wherein said combined score is indicative of efficacy from endocrine therapy of said patient, according to any of the methods as described herein.

### BRIEF DESCRIPTION OF THE DRAWINGS

FIG. 1 shows the deviation of EP scores generated by the alternative algorithm where BIRC5, AZGP1, and STC2 are not normalized. The graph illustrates a comparison of the alternative algorithm of the Example described herein from the EP score generated by the original EP score algorithm described in EP2553118B1. The original algorithm from the Y axis is dependent on the amount of input RNA as determined by the mean Ct value of the housekeeping genes as displayed on the X axis.

FIG. 2 shows the deviation of EP scores generated by the alternative algorithm where BIRC5, IL6ST, and STC2 are not normalized. The graph illustrates a comparison of the alternative algorithm of the Example described herein from the EP score generated by the original EP score algorithm described in EP2553118B1. The original algorithm from the Y axis is dependent on the amount of input RNA as determined by the mean Ct value of the housekeeping genes as displayed on the X axis.

FIG. 3 shows the deviation of EP scores generated by the alternative algorithm where IL6ST, DHCR7, and STC2 are not normalized. The graph illustrates a comparison of the alternative algorithm of the Example described herein from the EP score generated by the original EP score algorithm described in EP2553118B1. The original algorithm from the Y axis is dependent on the amount of input RNA as determined by the mean Ct value of the housekeeping genes as displayed on the X axis.

FIG. 4 shows the deviation of EP scores generated by the alternative algorithm where all eight EP genes are not normalized. The graph illustrates a comparison of the alternative algorithm of the Example described herein from the EP score generated by the original EP score algorithm described in EP2553118B1. The original algorithm from the Y axis is dependent on the amount of input RNA as determined by the mean Ct value of the housekeeping genes as displayed on the X axis.

### DETAILED DESCRIPTION

### Definitions

Unless defined otherwise, technical and scientific terms used herein have the same meaning as commonly understood by one of ordinary skill in the art to which this invention belongs.

The term “cancer” refers to uncontrolled cellular growth, and is not limited to any stage, grade, histomorphological feature, agressivity, or malignancy of an affected tissue or cell aggregation.

The term “predicting an outcome” of a disease, as used herein, is meant to include both a prediction of an outcome of a patient undergoing a given therapy and a prognosis of a patient who is not treated. The term “predicting an outcome” may, in particular, relate to the risk of a patient developing metastasis, local recurrence or death.

The term “prediction”, as used herein, relates to an individual assessment of the malignancy of a tumor, or to the expected survival rate (OAS, overall survival or DFS, disease free survival) of a patient, if the tumor is treated with a given therapy. In contrast thereto, the term “prognosis” relates to an individual assessment of the malignancy of a tumor, or to the expected survival rate (OAS, overall survival or DFS, disease free survival) of a patient, if the tumor remains untreated.

An “outcome” within the meaning of the present invention is a defined condition attained in the course of the disease. This disease outcome may e.g. be a clinical condition such as “recurrence of disease”, “development of metastasis”, “development of nodal metastasis”, development of distant metastasis”, “survival”, “death”, “tumor remission rate”, a disease stage or grade or the like.

A “risk” is understood to be a number related to the probability of a subject or a patient to develop or arrive at a certain disease outcome. The term “risk” in the context of the present invention is not meant to carry any positive or negative connotation with regard to a patient's wellbeing but merely refers to a probability or likelihood of an occurrence or development of a given condition.

The term “clinical data” relates to the entirety of available data and information concerning the health status of a patient including, but not limited to, age, sex, weight, menopausal/hormonal status, etiopathology data, anamnesis data, data obtained by in vitro diagnostic methods such as histopathology, blood or urine tests, data obtained by imaging methods, such as x-ray, computed tomography, MRI, PET, spect, ultrasound, electrophysiological data, genetic analysis, gene expression analysis, biopsy evaluation, intraoperative findings.

The term “node positive”, “diagnosed as node positive”, “node involvement” or “lymph node involvement” means a patient having previously been diagnosed with lymph node metastasis. It shall encompass both draining lymph node, near lymph node, and distant lymph node metastasis. This previous diagnosis itself shall not form part of the inventive method. Rather it is a precondition for selecting patients whose samples may be used for one embodiment of the present invention. This previous diagnosis may have been arrived at by any suitable method known in the art, including, but not limited to lymph node removal and pathological analysis, biopsy analysis, in-vitro analysis of biomarkers indicative for metastasis, imaging methods (e.g. computed tomography, X-ray, magnetic resonance imaging, ultrasound), and intraoperative findings.

In the context of the present invention a “biological sample” is a sample which is derived from or has been in contact with a biological organism. Examples for biological samples are: cells, tissue, body fluids, lavage fluid, smear samples, biopsy specimens, blood, urine, saliva, sputum, plasma, serum, cell culture supernatant, and others.

A “tumor sample” is a biological sample containing tumor cells, whether intact or degraded. The sample may be of any biological tissue or fluid. Such samples include, but are not limited to, sputum, blood, serum, plasma, blood cells (e.g., white cells), tissue, core or fine needle biopsy samples, cell-containing body fluids, urine, peritoneal fluid, and pleural fluid, liquor cerebrospinalis, tear fluid, or cells isolated therefrom. This may also include sections of tissues such as frozen or fixed sections taken for histological purposes or microdissected cells or extracellular parts thereof. A tumor sample to be analyzed can be tissue material from a neoplastic lesion taken by aspiration or punctuation, excision or by any other surgical method leading to biopsy or resected cellular material. Such comprises tumor cells or tumor cell fragments obtained from the patient. The cells may be found in a cell “smear” collected, for example, by a nipple aspiration, ductal lavage, fine needle biopsy or from provoked or spontaneous nipple discharge. In another embodiment, the sample is a body fluid. Such fluids include, for example, blood fluids, serum, plasma, lymph, ascitic fluids, gynecologic fluids, or urine but not limited to these fluids.

A “gene” is a set of segments of nucleic acid that contains the information necessary to produce a functional RNA product. A “gene product” is a biological molecule produced through transcription or expression of a gene, e.g., an mRNA, cDNA or the translated protein.

An “mRNA” is the transcribed product of a gene and shall have the ordinary meaning understood by a person skilled in the art. A “molecule derived from an mRNA” is a molecule which is chemically or enzymatically obtained from an mRNA template, such as cDNA.

The term “expression level” refers to a determined level of gene expression. This may be a determined level of gene expression as an absolute value or compared to a reference gene (e.g. a housekeeping gene), to the average of two or more reference genes, or to a computed average expression value (e.g. in DNA chip analysis) or to another informative gene without the use of a reference sample. The expression level of a gene may be measured directly, e.g. by obtaining a signal wherein the signal strength is correlated to the amount of mRNA transcripts of that gene or it may be obtained indirectly at a protein level, e.g., by immunohistochemistry, CISH, ELISA or RIA methods. The expression level may also be obtained by way of a competitive reaction to a reference sample. An expression value which is determined by measuring some physical parameter in an assay, e.g. fluorescence emission, may be assigned a numerical value which may be used for further processing of information.

A “reference pattern of expression levels” within the meaning of the invention shall be understood as being any pattern of expression levels that can be used for the comparison to another pattern of expression levels. In a preferred embodiment of the invention, a reference pattern of expression levels is, e.g., an average pattern of expression levels observed in a group of healthy individuals, diseased individuals, or diseased individuals having received a particular type of therapy, serving as a reference group, or individuals with good or bad outcome.

The term “mathematically combining expression levels”, within the meaning of the invention shall be understood as deriving a numeric value from a determined expression level of a gene and applying an algorithm to one or more of such numeric values to obtain a combined numerical value or combined score.

An “algorithm” is a process that performs some sequence of operations to produce information.

A “score” is a numeric value that was derived by mathematically combining expression levels using an algorithm. It may also be derived from expression levels and other information, e.g. clinical data. A score may be related to the outcome of a patient's disease. An EndoPredict® score (EP score) is a multivariate score for determining the risk of remote metastases in patients with an estrogen receptor-positive and HER2-negative primary mammary carcinoma under a sole adjuvant endocrine therapy. The EP score is a numerical measure of the relative risk that the tumor of the breast cancer patient examined with this EP score will develop remote metastases within 10 years.

A “discriminant function” is a function of a set of variables used to classify an object or event. A discriminant function thus allows classification of a patient, sample or event into a category or a plurality of categories according to data or parameters available from said patient, sample or event. Such classification is a standard instrument of statistical analysis well known to the skilled person. For example, a patient may be classified as “high risk” or “low risk”, “high probability of metastasis” or “low probability of metastasis”, “in need of treatment” or “not in need of treatment” according to data obtained from said patient, sample or event. Classification is not limited to “high vs. low”, but may be performed into a plurality of categories, grading or the like. Classification shall also be understood in a wider sense as a discriminating score, where e.g. a higher score represents a higher likelihood of distant metastasis, e.g., the (overall) risk of a distant metastasis. Examples for discriminant functions which allow a classification include, but are not limited to functions defined by support vector machines (SVM), k-nearest neighbors (kNN), (naive) Bayes models, linear regression models or piecewise defined functions such as, for example, in subgroup discovery, in decision trees, in logical analysis of data (LAD) and the like. In a wider sense, continuous score values of mathematical methods or algorithms, such as correlation coefficients, projections, support vector machine scores, other similarity-based methods, combinations of these and the like are examples for illustrative purpose.

The term “therapy modality”, “therapy mode”, “regimen” as well as “therapy regimen” refers to a timely sequential or simultaneous administration of anti-tumor, and/or anti vascular, and/or immune stimulating, and/or blood cell proliferative agents, and/or radiation therapy, and/or hyperthermia, and/or hypothermia for cancer therapy. The administration of these can be performed in an adjuvant and/or neoadjuvant mode. The composition of such “protocol” may vary in the dose of the single agent, timeframe of application and frequency of administration within a defined therapy window. Currently various combinations of various drugs and/or physical methods, and various schedules are under investigation.

The term “cytotoxic chemotherapy” refers to various treatment modalities affecting cell proliferation and/or survival. The treatment may include administration of alkylating agents, antimetabolites, anthracyclines, plant alkaloids, topoisomerase inhibitors, and other antitumor agents, including monoclonal antibodies and kinase inhibitors. In particular, the cytotoxic treatment may relate to a taxane treatment. Taxanes are plant alkaloids which block cell division by preventing microtubule function. The prototype taxane is the natural product paclitaxel, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

The term “endocrine treatment” or “hormonal treatment” (sometimes also referred to as “anti-hormonal treatment”) denotes a treatment which targets hormone signaling, e.g. hormone inhibition, hormone receptor inhibition, use of hormone receptor agonists or antagonists, use of scavenger- or orphan receptors, use of hormone derivatives and interference with hormone production. Particular examples are tamoxifene therapy which modulates signaling of the estrogen receptor, or aromatase treatment which interferes with steroid hormone production.

Tamoxifen is an orally active selective estrogen receptor modulator (SERM) that is used in the treatment of breast cancer and is currently the world's largest selling drug for that purpose. Tamoxifen is sold under the trade names Nolvadex, Istubal, and Valodex. However, the drug, even before its patent expiration, was and still is widely referred to by its generic name “tamoxifen.” Tamoxifen and Tamoxifen derivatives competitively bind to estrogen receptors on tumors and other tissue targets, producing a nuclear complex that decreases RNA synthesis and inhibits estrogen effects.

Steroid receptors are intracellular receptors (typically cytoplasmic) that perform signal transduction for steroid hormones. Examples include type I Receptors, in particular sex hormone receptors, e.g. androgen receptor, estrogen receptor, progesterone receptor; Glucocorticoid receptor, mineralocorticoid receptor; and type II Receptors, e.g. vitamin A receptor, vitamin D receptor, retinoid receptor, thyroid hormone receptor.

The term “hybridization-based method”, as used herein, refers to methods imparting a process of combining complementary, single-stranded nucleic acids or nucleotide analogues into a single double stranded molecule. Nucleotides or nucleotide analogues will bind to their complement under normal conditions, so two perfectly complementary strands will bind to each other readily. In bioanalytics, very often labeled, single stranded probes are used in order to find complementary target sequences. If such sequences exist in the sample, the probes will hybridize to said sequences which can then be detected due to the label. Other hybridization based methods comprise microarray and/or biochip methods. Therein, probes are immobilized on a solid phase, which is then exposed to a sample. If complementary nucleic acids exist in the sample, these will hybridize to the probes and can thus be detected. These approaches are also known as “array based methods.” Yet another hybridization based method is PCR, which is described below. When it comes to the determination of expression levels, hybridization based methods may for example be used to determine the amount of mRNA for a given gene.

An oligonucleotide capable of specifically binding sequences a gene or fragments thereof relates to an oligonucleotide which specifically hybridizes to a gene or gene product, such as the gene's mRNA or cDNA or to a fragment thereof. To specifically detect the gene or gene product, it is not necessary to detect the entire gene sequence. A fragment of about 20-150 bases will contain enough sequence specific information to allow specific hybridization.

The term “a PCR based method” as used herein refers to methods comprising a polymerase chain reaction (PCR). This is a method of exponentially amplifying nucleic acids, e.g. DNA by enzymatic replication in vitro. As PCR is an in vitro technique, it can be performed without restrictions on the form of DNA, and it can be extensively modified to perform a wide array of genetic manipulations. When it comes to the determination of expression levels, a PCR based method may for example be used to detect the presence of a given mRNA by (1) reverse transcription of the complete mRNA pool (the so called transcriptome) into cDNA with help of a reverse transcriptase enzyme, and (2) detecting the presence of a given cDNA with help of respective primers. This approach is commonly known as reverse transcriptase PCR (rtPCR). Moreover, PCR-based methods comprise e.g. real time PCR, and, particularly suited for the analysis of expression levels, kinetic or quantitative PCR (qPCR).

The term “Quantitative PCR” (qPCR)” refers to any type of a PCR method which allows the quantification of the template in a sample. Quantitative real-time PCR comprise different techniques of performance or product detection as for example the TaqMan technique or the LightCycler technique. The TaqMan technique, for examples, uses a dual-labelled fluorogenic probe. The TaqMan real-time PCR measures accumulation of a product via the fluorophore during the exponential stages of the PCR, rather than at the end point as in conventional PCR. The exponential increase of the product is used to determine the threshold cycle, CT, e.g., the number of PCR cycles at which a significant exponential increase in fluorescence is detected, and which is directly correlated with the number of copies of DNA template present in the reaction. The set up of the reaction is very similar to a conventional PCR, but is carried out in a real-time thermal cycler that allows measurement of fluorescent molecules in the PCR tubes. Different from regular PCR, in TaqMan real-time PCR a probe is added to the reaction, e.g., a single-stranded oligonucleotide complementary to a segment of 20-60 nucleotides within the DNA template and located between the two primers. A fluorescent reporter or fluorophore (e.g., 6-carboxyfluorescein, acronym: FAM, or tetrachlorofluorescein, acronym: TET) and quencher (e.g., tetramethylrhodamine, acronym: TAMRA, of dihydrocyclopyrroloindole tripeptide ‘black hole quencher’, acronym: BHQ) are covalently attached to the 5′ and 3′ ends of the probe, respectively. The close proximity between fluorophore and quencher attached to the probe inhibits fluorescence from the fluorophore. During PCR, as DNA synthesis commences, the 5′ to 3′ exonuclease activity of the Taq polymerase degrades that proportion of the probe that has annealed to the template. Degradation of the probe releases the fluorophore from it and breaks the close proximity to the quencher, thus relieving the quenching effect and allowing fluorescence of the fluorophore. Hence, fluorescence detected in the real-time PCR thermal cycler is directly proportional to the fluorophore released and the amount of DNA template present in the PCR.

By “array” or “matrix” an arrangement of addressable locations or “addresses” on a device is meant. The locations can be arranged in two dimensional arrays, three dimensional arrays, or other matrix formats. The number of locations can range from several to at least hundreds of thousands. Most importantly, each location represents a totally independent reaction site. Arrays include but are not limited to nucleic acid arrays, protein arrays and antibody arrays. A “nucleic acid array” refers to an array containing nucleic acid probes, such as oligonucleotides, nucleotide analogues, polynucleotides, polymers of nucleotide analogues, morpholinos or larger portions of genes. The nucleic acid and/or analogue on the array is preferably single stranded. Arrays wherein the probes are oligonucleotides are referred to as “oligo¬nucleotide arrays” or “oligonucleotide chips.” A “microarray,” herein also refers to a “biochip” or “biological chip”, an array of regions having a density of discrete regions of at least about 100/cm2, and preferably at least about 1000/cm2.

“Primer pairs” and “probes” within the meaning of the invention shall have the ordinary meaning of this term which is well known to the person skilled in the art of molecular biology. In a preferred embodiment of the invention “primer pairs” and “probes” shall be understood as being polynucleotide molecules having a sequence identical, complementary, homologous, or homologous to the complement of regions of a target polynucleotide which is to be detected or quantified. In yet another embodiment, nucleotide analogues are also comprised for usage as primers and/or probes. Probe technologies used for kinetic or real time PCR applications could be e.g. TaqMan® systems obtainable at Applied Biosystems, extension probes such as Scorpion® Primers, Dual Hybridisation Probes, Amplifluor® obtainable at Chemicon International, Inc, or Minor Groove Binders.

“Individually labeled probes”, within the meaning of the invention, shall be understood as being molecular probes comprising a polynucleotide, oligonucleotide or nucleotide analogue and a label, helpful in the detection or quantification of the probe. Preferred labels are fluorescent molecules, luminescent molecules, radioactive molecules, enzymatic molecules and/or quenching molecules.

“Arrayed probes”, within the meaning of the invention, shall be understood as being a collection of immobilized probes, preferably in an orderly arrangement. In a preferred embodiment of the invention, the individual “arrayed probes” can be identified by their respective position on the solid support, e.g., on a “chip”.

When used in reference to a single-stranded nucleic acid sequence, the term “substantially homologous” refers to any probe that can hybridize (i.e., it is the complement of) the single-stranded nucleic acid sequence under conditions of low stringency as described above.

To determine an EP score, the relative RNA expression of eight genes is measured, and their measured values are used for calculation by means of a discriminate function. The RNA expression can be determined with any technical method suitable for quantifying RNA. Because of its high analytical sensitivity and the possibility to analyze even small RNA fragments obtained in the recovery of tumor RNA from formalin-fixed and paraffin-embedded breast cancer tissue, the quantitative polymerase chain reaction with previous reverse transcription (RT-qPCR) is a suitable technical mode for performing the analysis. However, microarray analysis or RNA sequencing are equally suitable for determining an EP score. The EndoPredict® score and the necessary technical method for determining it is described in Filipits et al. (2011) and in EP 2 553 118, both of which are incorporated herein by reference.

For the described calculation of the EP score, the measured values of the mRNA expression of a total of 11 genes are used. Among these, eight are so-called informative genes, whose expression level in combination correlates with the further course of the disease. The three remaining genes are reference genes, sometimes referred to as “normalization genes”.

The measured value obtained upon performing RT-qPCR, which inversely correlates with the quantity of RNA present in the analyzed sample, is the Ct value. It indicates after how many amplification cycles a sufficient amount of the PCR probe has been enzymatically degraded, so that the thus achieved reduction of the fluorescence quenching of the PCR dye by the PCR quencher is sufficient to be able to measure the fluorescence of the PCR dye. Therefore, a high Ct value in RT-qPCR is an indicator of a small amount of RNA to be analyzed in a sample.

The level of the Ct value depends on the concentration of the analyzed RNA in the sample, and also primarily on the total amount of RNA in the sample. However, especially in the analysis of a tissue sample, it is difficult to precisely define the amount of analyzed tissue and thus to be able to calculate a concentration in the tissue. This is mainly because tissues are mostly heterogeneous. The water content above all, but also the lipid content or the proportion of non-cellular components, can vary significantly. Thus, variations in the analysis of the RNA amounts of different genes in human or animal tissue often rather reflect the variation of the amount of the cellular fraction of the tissue subjected to in the analysis than the actually interesting biological differences between different tissue samples. In addition, the result of an RNA quantification is often substantially affected by the integrity of the RNA to be analyzed and by the amplification efficiency of the reagents employed. Therefore, the Ct values obtained in the RNA analysis of tissue are often primarily the product of different experimental factors, and to a lesser extent caused by the actually examined biological differences between the analyzed samples. Thus, if it is desired to measure the concentration of RNA in the cells of a tissue sample, the Ct value as a raw measured value of RT-qPCR is usually unsuitable.

Therefore, in order to be able to compare the RNA concentrations in two different tissue samples in a reasonable way, the Ct values must always be normalized on the basis of an invariant reference quantity. The obvious approach would be to normalize the Ct value on the basis of a particular amount of tissue, for example, one milligram or one microgram. However, because of the heterogeneity of the tissue, this method is practicable only to a very limited degree and is rarely used. The most common method in RT-qPCR is the normalization of the Ct values of the analyzed RNA transcripts (genes of interest or GOI) on the basis of the Ct value of one or more other, invariant genes in the same sample. These invariant genes are mostly referred to as reference or normalization genes, sometimes also as “housekeeper genes.” The invariance of the RNA expression of the normalization gene under the measuring conditions is the primary requirement demanded of a normalization gene. A variability of the amount of the RNA transcript of the normalization gene would reduce the purpose of normalization. A variant normalization gene has the consequence that the allegedly “normalized” Ct value of a “gene of interest” is actually not normalized. In this case, it depends on factors other than the transcript concentration of the gene of interest. Therefore, the normalization of a “gene of interest” using a variant gene or the correspondingly variant average of several non-variant genes is not a normalization at all, because the correspondingly formed “two-gene ratio” does not allow conclusions to be made on the transcript quantity of the “gene of interest.”

Because the invariance of a single gene is often difficult to ensure, the expression level of the RNA of several reasonably invariant genes are averaged in practice, expecting that the average of these genes exhibits a lower biological variance than that of the RNA concentration of each individual normalization gene.

An alternative normalization method is to average the RNA expression level of a large number of genes, including genes known to be variant, expecting that the average of the variance of the expression of these many genes will cancel out from examined sample to examined sample, and that the average of the expression of these genes will therefore be equal in all examined samples. This method of normalization is sometimes referred to as “global scaling.”

In any event, the RNA quantity of the “gene of interest” is expressed relative to the RNA quantity of one invariant gene, to the average of the RNA quantities of some invariant genes, or to the average of a large number of arbitrarily chosen genes. This is usually done by dividing the RNA quantity of the “gene of interest” by the quantity of RNA of the reference gene, or by the average of the RNA quantities of the reference genes. Because there is a logarithmic relationship between the Ct value and the RNA quantity, the normalization is then performed by subtracting the Ct values. This method is referred to as a delta-CT method. The normalized Ct value obtained is usually referred to as a delta-CT value.

In this way, the described EP score is calculated in two steps from the Ct values of the RNA molecules measured for the determination of the EP score: at first, the eight informative genes are normalized against the average of three invariant reference genes, and then the delta-Ct values of the eight informative genes are linearly combined.

A consequence of this approach is the fact that the transcript quantities of a total of 11 genes must be analyzed for determining the EndoPredict® score (EOP score) consisting of 8 genes. Thus, about a quarter of the cost and expenses of the determination of the EndoPredict® score is required for the determination of the transcripts necessary for normalizing the measured values. Thus, it is the object of the present invention to provide a method for determining the EP score simply but reliably without having to determine the RNA quantity of normalization genes.

According to the invention, this object is achieved by a method for predicting a result relating to breast cancer in an estrogen receptor-positive and HER2-negative tumor in a breast cancer patient, the method comprising:

(a) determining the RNA expression levels of four or more of the following 8 genes in a tumor sample from the patient: UBE2C, BIRC5, DHCR7, STC2, AZGP1, RBBP8, IL6ST and MGP;
(b) mathematically combining the expression level values for the genes of the mentioned set, the values having been determined in the tumor sample, to obtain a combined score, the combined score indicating a prognosis for the patient, wherein the RNA expression levels have at least in part not been normalized before the mathematical combination.

In some embodiments the four or more genes are BIRC5, UBE2C, RBBP8, and IL6ST. Additional embodiments of the four of more genes can include any of the biomarker panels described in Table 1.

TABLE 1

| | |
|---|---|
| Panel 1 | BIRC5, UBE2C, RBBP8, and IL6ST |
| Panel 2 | BIRC5, UBE2C, RBBP8, IL6ST, and DHCR7 |
| Panel 3 | BIRC5, UBE2C, RBBP8, IL6ST, and AZGP1 |
| Panel 4 | BIRC5, UBE2C, RBBP8, IL6ST, and MGP |
| Panel 5 | BIRC5, UBE2C, RBBP8, IL6ST, and STC2 |
| Panel 6 | BIRC5, UBE2C, RBBP8, IL6ST, DHCR7, and AZGP1 |
| Panel 7 | BIRC5, UBE2C, RBBP8, IL6ST, DHCR7, and MGP |
| Panel 8 | BIRC5, UBE2C, RBBP8, IL6ST, DHCR7, and STC2 |
| Panel 9 | BIRC5, UBE2C, RBBP8, IL6ST, AZGP1, and MGP |
| Panel 10 | BIRC5, UBE2C, RBBP8, IL6ST, AZGP1, and STC2 |
| Panel 11 | BIRC5, UBE2C, RBBP8, IL6ST, MGP, and STC2 |
| Panel 12 | BIRC5, UBE2C, RBBP8, IL6ST, DHCR7, AZGP1, and MGP |
| Panel 13 | BIRC5, UBE2C, RBBP8, IL6ST, DHCR7, AZGP1, and STC |
| Panel 14 | BIRC5, UBE2C, RBBP8, IL6ST, DHCR7, MGP, and STC |
| Panel 15 | BIRC5, UBE2C, RBBP8, IL6ST, AZGP1, MGP, and STC |
| Panel 16 | BIRC5, UBE2C, RBBP8, IL6ST, DHCR7, AZGP1, MGP, |
| | and STC |

It is not always optimal to normalize the RNA quantity (transcript quantity), i.e., the Ct value, of a “gene of interest” on the basis of the RNA quantity of another “gene of interest” or of the average of some or all “genes of interest.” The transcript quantities of the “genes of interest” are of course highly different among the samples because the genes in the EP score were purposefully selected to reflect the biological variance of different samples. However, to relate a variant transcript quantity to another variant transcript quantity might not be expedient, as described above, because this still would not allow one to compare transcript quantities of a “gene of interest” among the samples.

As a result, the measurement of genes in addition to the eight “genes of interest” in the EP score can be omitted only if the normalization of the “genes of interest” can be successfully dispensed with altogether.

The method according to the invention is based on the fact that the Ct values, which, are raw values, do not exclusively reflect the RNA quantities of the genes determined for the EP score, as described above, nevertheless are not normalized, and also remain unnormalized in the further course of the calculation of the EP score. Then, the comparability of different EP scores determined on different tumor samples is accordingly not obtained by normalizing the Ct values of the genes from which the EP score is calculated, making them comparable, but the comparability is advantageously reached on the level of the EP score.

This is further explained by means of the following technical measure:

The eight genes of interest of the EP score are first normalized on the basis of the average of three reference genes, and the EP score is represented as a linear combination of the total of 11 measured Ct values according to equation (3) (see below). Surprisingly, when the method according to the invention is applied to the EndoPredict® method, in particular, it results that the sum of the linear coefficients of the eight “genes of interest” according to equation (6) is relatively small, so that the corresponding term can therefore be neglected as a good approximation. A new EP score is obtained (equation (8)), which, although not identical with previous, conventionally calculated scores (Filipits et al.), deviates only slightly therefrom and does not deteriorate the prognostic value of the assay, thus being clinically irrelevant. An advantage of the method according to the invention is the fact that no reference genes need to be measured for calculating the new EP score: this simplifies the production of test kits (PCR primers and probes) and the performance of the test on the user's part.

Indeed, the individual transcript amounts of the individual genes are no longer normalized in the method according to the invention. Therefore, normalized expression levels are no longer derivable even within the calculation of the EP score. Thus, the comparability of different EP scores from different samples is no longer derived from the comparability of the Ct values (these are actually not comparable among the samples), but from the fact that the sum of the coefficients used for the linear combination of the Ct values is not substantially different from zero. As a consequence, although the measurement of one and the same tissue sample may yield significantly different raw Ct values of all individual genes because of different starting quantities and different RNA qualities, the sum of all these weighted individual genes is nevertheless essentially constant. For this reason, a new EP score that is well comparable among the samples is obtained despite a lack of normalization of the individual genes.

The normalization-free calculation of the EP score cannot be derived mathematically from the already published calculation of the EP score with normalization. This is because the two kinds of calculation are not equivalent. Especially in EndoPredict®, the possibility to dispense with measuring the normalization genes results from the fact that the sum of the coefficients on the linear combination of the delta Ct values is not large, because the terms are in part positive and in part negative numbers. Thus, setting this sum to zero is a mistake in strictly mathematical terms. However, the produced mistake is small and acceptable especially before the background of the imprecision of the measured values. However, it allows a greatly simplified and yet reliable determination in the specific case of the EP score.

The first step in the calculation of the EP score is the determination of delta-Ct values. The following definition is used:

Δᵢ=20−xᵢ+r (1)

In this equation, Δᵢ is the delta-Ct value of the “gene of interest” i, xᵢ is the Ct value of gene i, and r is the average of the Ct values of the three reference genes. The EP score uses eight informative genes (BIRC5, RBBP8, UBE2C, IL6ST, AZGP1, DHCR7, MGP and STC2) and three reference genes (CALM2, OAZ1 and RPL37A).

In the second step, the eight delta-Ct values are calculated into one score.

$$EP = {\sum }_{i = 1}^{8} {c}_{i} {Δ}_{i} \qquad ( 2 )$$

Herein, EP is the (unscaled) EP score, and cᵢ is the linear coefficient for the informative gene i. As already published by Filipits, the linear coefficients are:

TABLE 2

| i | Gene name | cᵢ |
|---|---|---|
| 1 | BIRC5 | 0.407753 |
| 2 | RBBP8 | −0.347558 |
| 3 | UBE2C | 0.388326 |
| 4 | IL6ST | −0.305020 |
| 5 | AZGP1 | −0.264064 |
| 6 | DHCR7 | 0.394019 |
| 7 | MGP | −0.183334 |
| 8 | STC2 | −0.146689 |

The third and last step of the calculation of the EP score consists in a scaling and limiting step. However, it is not relevant to the result and merely transfers the results to a more intuitive scale. This step will be ignored in the further considerations.

In order to calculate EP directly from the Ct values xᵢ, equation (1) is substituted into equation (2) to obtain equation (3).

$$EP = {\sum }_{i = 1}^{8} {c}_{i} ( 20 - {x}_{i} + r ) \qquad ( 3 )$$

Now, the Ct values of the informative genes x₁, . . . , x₈ can be separated from the average of the Ct values of the reference genes r by factoring:

$$EP = {\sum }_{i = 1}^{8} - {c}_{i} {x}_{i} + ( 20 + r ) {\sum }_{i = 1}^{8} {c}_{i} \qquad ( 4 )$$

The second factor in the second addend can be calculated with the aid of Table 2.

$${\sum }_{i = 1}^{8} {c}_{i} = - 0.056567 \qquad ( 5 )$$

Thus, in the special case of the coefficients in EndoPredict®, the absolute value of this sum is relatively small (significantly smaller than any of its addends) and therefore, as a special case, allows the following surprising approximation of a new EP score:

$$= {\sum }_{i = 1}^{8} - {c}_{i} {x}_{i} + ( 20 + \tilde{r} ) {\sum }_{i = 1}^{8} {c}_{i} \qquad ( 6 )$$

Here, only two variables were replaced as compared to equation (4): [symbol] designates the new approximated EP score, and r̅ designates a constant, which is determined below. In particular, r̅ (unlike r) is not dependent on measured values of the patient sample in question.

Now, after the definition of the approximated EP score according to equation (6), what is interesting above all is the difference between the new EP score and the previous EP score according to equation (4). It is obtained by subtracting equations (6) and (4) to give equation (7).

$$- EP = ( \tilde{r} - r ) {\sum }_{i = 1}^{8} {c}_{i} = - 0.056567 \cdot ( \tilde{r} - r ) \qquad ( 7 )$$

On the basis of this equation, it is clear that the alteration of the score, i.e., [symbol] −EP, can be kept small if the constant r̅ is selected to be close to r.

Empirical studies showed that r is typically within the interval of from 19 to 27. This value results from the RNA quantity that can typically be isolated from a tumor sample. In practice, a value of from r̅=21 to 25, preferably r̅=23, suggests itself for r̅. Thus, |r̅−r|≤4 would apply, and the deviation | [symbol] −EP|≤0.226270 would be acceptably small (this means a maximum variation of 0.339406 for the EP score scaled according to Filipits et al.; this value is thus smaller than half the width of the 95% confidence interval of the measuring accuracy of about 0.5). In accordance with the above and because of the small value of the sum over cᵢ, there is obtained as an approximation for the calculation of the EP score according to equation (6):

$$\overline{EP} = {\sum }_{i = 1}^{8} - {c}_{i} {x}_{i} - 2.432381 \qquad ( 8 )$$

Thus, an approximated form of the EP score, which is not completely invariant towards variations of the RNA input amount in accordance with the omission of normalization, can actually be derived according to equation (8). However, it allows a clearly simpler performing of the test. Because of the omission of normalization, 3 of the 11 RNA measurements can be omitted. Thus, because of the reduced number of measurements necessary for the determination of the EP score, the overall precision of the measurement and thus the repeatability of the overall result is also improved.

From the disclosure, it can be seen that it is not only possible to perform an approximate calculation of the EP score according to equation (8) by normalizing none of the RNA expression levels of any gene. It is also possible to calculate part of an approximate EP score from the normalized value of the RNA expression of some genes by analogy with equation (3), and to calculate some other part of the EP score from the unnormalized RNA expression levels of the remaining genes by analogy with equation (6) according to equation (9):

$$= {\sum }_{i = 1}^{k} - {c}_{i} {x}_{i} + ( 20 + \tilde{r} ) {\sum }_{i = 1}^{k} {c}_{i} + {\sum }_{i = k + 1}^{8} - {c}_{i} {x}_{i} + ( 20 + r ) {\sum }_{i = k + 1}^{8} {c}_{i} \qquad ( 9 )$$

wherein k must be a natural number from 1 to 6. Further, it is important for the genes whose measuring results are included in the modified EP score without normalization to be selected in such a way that the absolute value of the sum of linear coefficients cᵢ corresponding to such genes according to Table 2 is as low as possible, preferably lower than 0.06. Thus, suitable gene combinations that can be included in the modified EP score without normalization are, for example, BIRC5, AZGP1, STC2 (sum over cᵢ equals −0.003) or BIRC5 and IL6ST and STC2 (sum over cᵢ equals −0.043956) or IL6ST and DHCR7 and STC2 (sum over cᵢ equals −0.05769). The respectively remaining genes of the EP score would then be included in the modified EP score in an individually normalized form.

Absolute coefficients are thus for proliferation genes: BIRC5 (coefficient: 0.41), UBE2C (0.39), DHCR7 (0.39) and differentiation/ER signalling genes: RBBP8 (0.35), IL6ST (0.31), AZGP1 (0.26), MGP (0.18), STC2 (0.15).

### Example

Aspects of the present teachings can be further understood in light of the following examples, which should not be construed as limiting the scope of the present teachings in any way.

This example demonstrates the ability to determine an EndoPredict® EP score (an “EP score”) either without having to determine the RNA quantity of normalization genes, or by determining RNA quantities using partial normalization.

Total RNA was extracted from 881 samples of patients with ER+, HER2− primary breast cancer samples was extracted with a Siemens, silica bead-based and fully automated isolation method for RNA from one 10 μm whole FFPE tissue section on a Hamilton MICROLAB STARlet liquid handling robot (17). The robot, buffers and chemicals were part of a Siemens VERSANT® kPCR Molecular System (Siemens Healthcare Diagnostics, Tarrytown, N.Y.; not commercially available in the USA). Briefly, 150 μl FFPE buffer (Buffer FFPE, research reagent, Siemens Healthcare Diagnostics) were added to each section and incubated for 30 minutes at 80° C. with shaking to melt the paraffin. After cooling down, proteinase K was added and incubated for 30 minutes at 65° C. After lysis, residual tissue debris was removed from the lysis fluid by a 15 minutes incubation step at 65° C. with 40 μl silica-coated iron oxide beads. The beads with surface-bound tissue debris were separated with a magnet and the lysates were transferred to a standard 2 ml deep well-plate (96 wells). There, the total RNA and DNA was bound to 40 μl unused beads and incubated at room temperature. Chaotropic conditions were produced by the addition of 600 μl lysis buffer. Then, the beads were magnetically separated and the supernatants were discarded. Afterwards, the surface-bound nucleic acids were washed three times followed by magnetization, aspiration and disposal of supernatants. Afterwards, the nucleic acids were eluted by incubation of the beads with 100 μl elution buffer for 10 minutes at 70° C. with shaking. Finally, the beads were separated and the supernatant incubated with 12 μl DNase I Mix (2 μL DNase I (RNase free); 10 μl 10× DNase I buffer; Ambi-on/Applied Biosystems, Darmstadt, Germany) to remove contaminating DNA. After incubation for 30 minutes at 37° C., the DNA-free total RNA solution was aliquoted and stored at −80° C. or directly used for mRNA expression analysis by reverse transcription kinetic PCR (RTkPCR). All the samples were analyzed with one-step RT-kPCR in an ABI PRISM® 7900HT (Applied Biosystems, Darmstadt, Germany). The SuperScript® III Platinum® One-Step Quantitative RT-PCR System with ROX (6-carboxy-X-rhodamine) (Invitrogen, Karlsruhe, Germany) was used according to the manufacturer's instructions. Respective probes and primers are described previously (EP 2 553 118 B1). The PCR conditions were as follows: 30 minutes at 50° C., 2 minutes at 95° C. followed by 40 cycles of 15 seconds at 95° C. and 30 seconds at 60° C. All the PCR assays were performed in triplicate.

Following extraction of RNA and assessment of mRNA levels of the 8 EP genes-of-interest BIRC5, UBE2C, DHCR7, RBBP8, IL6ST, AZGP1, MGP, and STC2, as well as the three reference genes RPL37A, CALM2, and OAZ1 by RT-PCR, alternative algorithms were applied that lacked normalization of all eight EP genes or different subsets of EP genes. The first step in the calculation of the EP score was the determination of delta-Ct values. The following definition was used:

Δᵢ=20−xᵢ+r (1)

In this equation, Δᵢ is the delta-Ct value of the “gene of interest” i, xᵢ is the Ct value of gene i, and r is the average of the Ct values of the three reference genes as described herein

In the second step, the eight delta-Ct values are calculated into one score.

$$EP = {\sum }_{i = 1}^{8} {c}_{i} {Δ}_{i} \qquad ( 2 )$$

Herein, EP is the (unscaled) EP score, and cᵢ is the linear coefficient for the informative gene i. The linear coefficients were those used as published by Filipits (2011).

In order to calculate EP directly from the Ct values xᵢ, equation (1) was substituted into equation (2) to obtain equation (3).

$$EP = {\sum }_{i = 1}^{8} {c}_{i} ( 20 - {x}_{i} + r ) \qquad ( 3 )$$

Ct values of the informative genes x₁, . . . , x₈ were then separated from the average of the Ct values of the reference genes r by factoring:

$$EP = {\sum }_{i = 1}^{8} - {c}_{i} {x}_{i} + ( 20 + r ) {\sum }_{i = 1}^{8} {c}_{i} \qquad ( 4 )$$

The second factor in the second addend was then calculated using the linear coefficients.

$${\sum }_{i = 1}^{8} {c}_{i} = - 0.056567 \qquad ( 5 )$$

Thus, the absolute value of this sum was relatively small, thus allowing approximation of a new EP score:

$$= {\sum }_{i = 1}^{8} - {c}_{i} {x}_{i} + ( 20 + \tilde{r} ) {\sum }_{i = 1}^{8} {c}_{i} \qquad ( 6 )$$

Here, only two variables were replaced as compared to equation (4): [symbol] designates the new approximated EP score, and r̅ designates a constant, which designates a constant equaling 23 as described in the specification herein. In particular, r̅ (unlike r) is not dependent on measured values of the patient sample in question.

Now, after the definition of the approximated EP score according to equation (6), the difference between the new EP score and the previous EP score was obtained by subtracting equations (6) and (4) to give equation (7).

$$= EP = ( \overline{r} - r ) {\sum }_{i = 1}^{8} {c}_{i} = - 0.056567 \cdot ( \overline{r} - r ) \qquad ( 7 )$$

On the basis of this equation, it was clear that the alteration of the score, i.e., [symbol] −EP, can be kept small if the constant r̅ is selected to be close to r.

Because of the small value of the sum over cᵢ, an approximation for the calculation of the EP score was obtained according to equation (6) with r̅=23:

$$\overline{EP} = {\sum }_{i = 1}^{8} - {c}_{i} {x}_{i} - 2.432381 \qquad ( 8 )$$

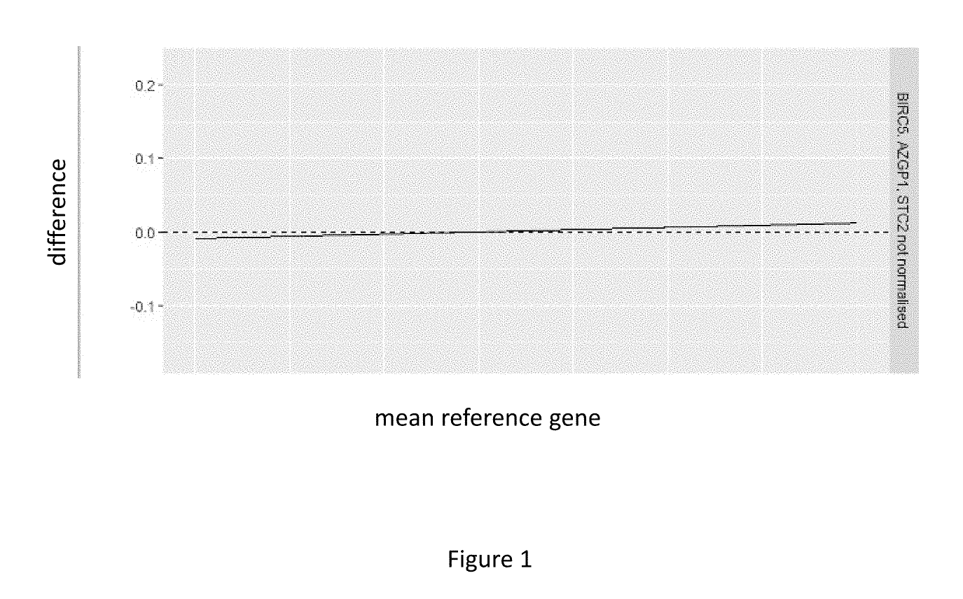

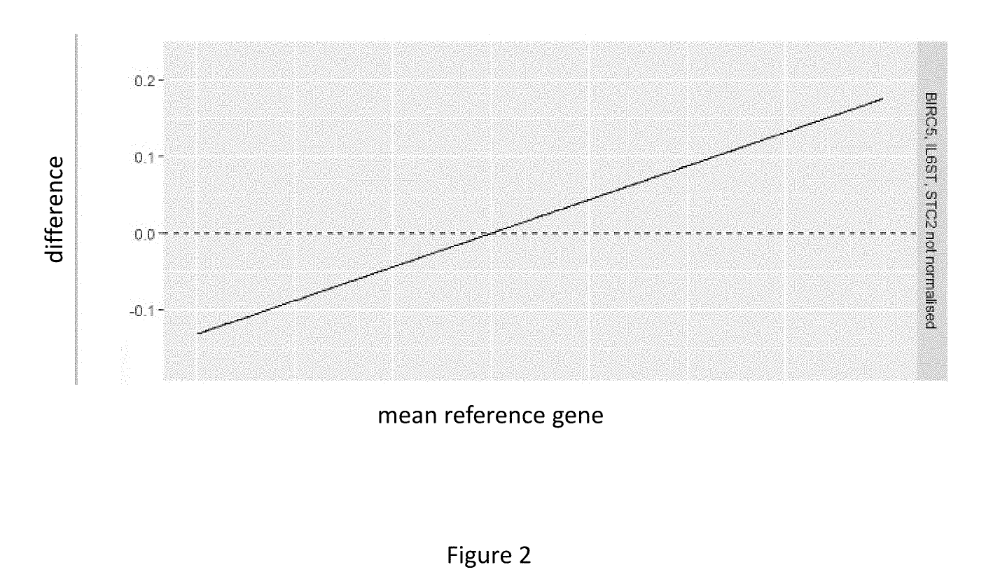

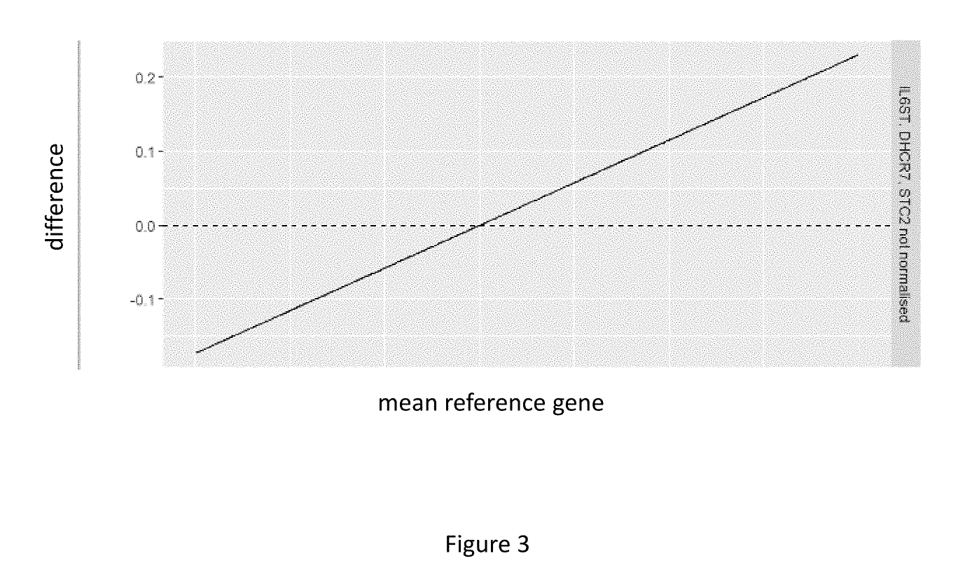

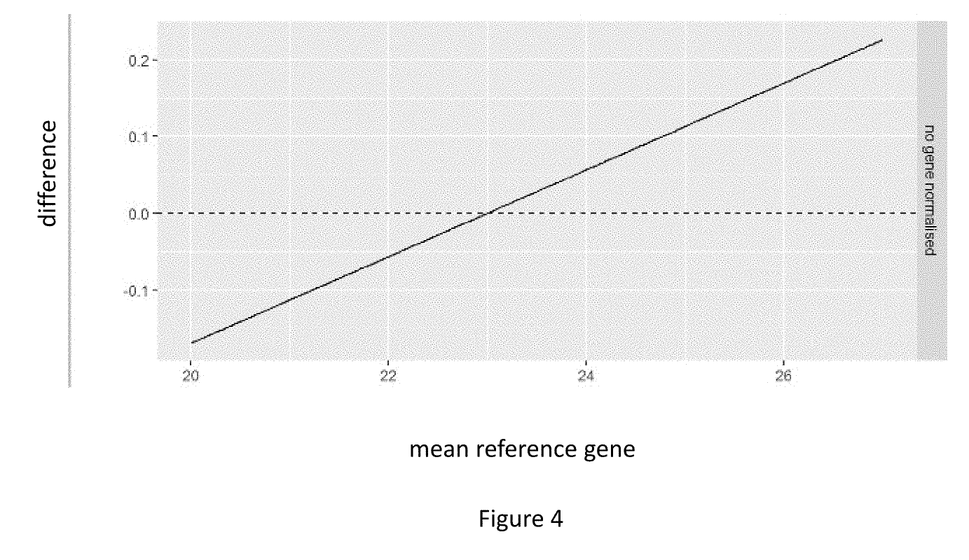

It was also possible to calculate part of an approximate EP score from the normalized value of the RNA expression of some genes by analogy with equation (3), and to calculate some other part of the EP score from the unnormalized RNA expression levels of the remaining genes by analogy with equation (6) according to equation (9):

$$= {\sum }_{i = 1}^{k} - {c}_{i} {x}_{i} + ( 20 + \tilde{r} ) {\sum }_{i = 1}^{k} {c}_{i} + {\sum }_{i = k + 1}^{8} - {c}_{i} {x}_{i} + ( 20 + r ) {\sum }_{i = k + 1}^{8} {c}_{i} \qquad ( 9 )$$

wherein k must be a natural number from 1 to 6. Thus, suitable gene combinations that can be included in the modified EP score without normalization are, for example, BIRC5, AZGP1, STC2 (sum over cᵢ equals −0.003) (FIG. 1) or BIRC5 and IL6ST and STC2 (sum over cᵢ equals −0.043956) (FIG. 2) or IL6ST and DHCR7 and STC2 (sum over cᵢ equals −0.05769) (FIG. 3). The respectively remaining genes of the EP score would then be included in the modified EP score in an individually normalized form. FIG. 4 demonstrates the lack of normalization of all eight EP genes.

1. A method for predicting a result relating to breast cancer in an estrogen receptor-positive and HER2-negative tumor in a breast cancer patient, the method comprising:
(a) determining the RNA expression levels of at least 4 of the following 8 genes in a tumor sample from the patient: UBE2C, BIRC5, DHCR7, STC2, AZGP1, RBBP8, IL6ST and MGP;
(b) mathematically combining the expression level values for the genes of the mentioned set, the values having been determined in the tumor sample, to obtain a combined score, the combined score indicating a prognosis for the patient, wherein the RNA expression level values have at least in part not been normalized before the mathematical combination.

2. The method according to claim 1, wherein the at least 4 genes are BIRC5, UBE2C, RBBP8, and IL6ST.

3. The method according to claim 1, wherein the at least 4 genes are any of the panels described in Table 1.

4. The method according to any of claims 1 to 3, wherein said mathematically combining the expression levels is effected by using the formula
$$= {\sum }_{i = 1}^{8} - {c}_{i} {x}_{i} - 2.432381$$
$$or$$
$$= {\sum }_{i = 1}^{k} - {c}_{i} {x}_{i} + ( 20 + \tilde{r} ) {\sum }_{i = 1}^{k} {c}_{i} + {\sum }_{i = k + 1}^{8} - {c}_{i} {x}_{i} + ( 20 + r ) {\sum }_{i = k + 1}^{8} {c}_{i} .$$

5. The method according to any one of claims 1 to 4, wherein said patient has received endocrine therapy or is contemplated to receive endocrine treatment.

6. The method according to any one of claims 1 to 5 wherein a risk of developing breast cancer recurrence or cancer-related death is predicted.

7. Method according to any of claims 1 to 6, wherein said expression level is determined as a Messenger-RNA expression level.

8. Method according to claim 7, wherein said expression level is determined by at least one of
a PCR based method,
a microarray based method, and
a hybridization based method.

9. Method of any one of the preceding claims, wherein said determination of expression levels is in a formalin-fixed paraffin embedded tumor sample or in a fresh-frozen tumor sample.

10. Method of any one of the preceding claims, wherein one, two or more thresholds are determined for said combined score, that discriminate into high and low risk, high, intermediate and low risk, or more risk groups by applying the threshold on the combined score.

11. Method of any of claims 1 to 10, wherein a high combined score is indicative of benefit from cytotoxic chemotherapy.

12. Method of any one of the preceding claims, wherein information regarding nodal status of the patient is processed in the step of mathematically combining expression level values for the genes to yield a combined score.

13. Method of claim 12, wherein said information regarding nodal status is a numerical value if said nodal status is negative and said information is a different numerical value if said nodal status positive and a different or identical number if said nodal status is unknown.

14. A kit for performing a method of any of claims 1 to 13, said kit comprising a set of oligonucleotides capable of specifically binding sequences or to sequences of fragments of the genes in a combination of genes, wherein
said combination comprises determining the RNA expression levels of at least 4 of the following 8 genes in a tumor sample from the patient: UBE2C, BIRC5, DHCR7, STC2, AZGP1, RBBP8, IL6ST and MGP.

15. The kit according to claim 14, wherein the at least 4 genes are BIRC5, UBE2C, RBBP8, and IL6ST.

16. The kit according to claim 14, wherein the at least 4 genes are any of the panels described in Table 1.

17. A computer program product capable of processing values representative of expression levels of a set of genes, mathematically combining said values to yield a combined score, wherein said combined score is indicative of efficacy from endocrine therapy of said patient, according to the methods of any of claims 1 to 13.
